(19) United States
(12) Patent Application Publication
(10) Pub. No.: US 2013/0053672 A1
(43) Pub. Date: Feb. 28, 2013

(54) SYSTEM AND METHOD FOR THE USE OF A PORTABLE DEVICE TO PROVIDE NEURAL FEEDBACK TREATMENT AND ANY TYPE OF TRANS-CRANIAL DIRECT CURRENT STIMULATION AND SERVICES

(71) Applicant: Joan SANGER, Waterloo (CA)

(72) Inventor: Joan SANGER, Waterloo (CA)

(21) Appl. No.: 13/592,321

(22) Filed: Aug. 22, 2012

Related U.S. Application Data
(60) Provisional application No. 61/526,019, filed on Aug. 22, 2011.

(51) Int. Cl. A61B 5/0482; A61B 5/0478

(52) U.S. Cl. 600/383

(57) ABSTRACT

A neural feedback system uses a headset with electrodes for detecting brain waves that is coupled with a portable device such as a smart phone. The brain waves are monitored by a software application running on the mobile device in coordination with a server, or independently on the mobile device, to output a measure of the brain waves that correlates with a user's level of brain activity. When the output matches or exceeds predetermined conditions, the wearer of the headset receives predetermined messages, prompts or other media support via the mobile device which are tailored to the individual.

A neural feedback system uses a headset with electrodes for detecting brain waves that is coupled with a portable device such as a smart phone. The brain waves are monitored by a software application running on the mobile device in coordination with a server, or independently on the mobile device, to output a measure of the brain waves that correlates with a user's level of brain activity. When the output matches or exceeds predetermined conditions, the wearer of the headset receives predetermined and/or individualized messages, prompts or other media support via the mobile device which are tailored to the individual.

### SUMMARY OF THE INVENTION

The present invention addresses at least some of the shortcomings of the previous art by providing a comprehensive, accessible and easy-to-use neural feedback system to be delivered via a portable device that uses EEG, detected through a headband or headset. Electrodes on the headset detect the brain waves and provide an output data stream of the detected brain waves that is received via a mobile computer processor, such as a smart phone. With this approach, the brain wave generated output can be monitored through software running on the mobile device to determine if the brain waves match predetermined levels, exceed such levels or suggest brain wave patterns that have been predetermined to generate or suggest a responsive action by the user. The responsive action can be then taken by the user through the mobile device which can provide the selection of media support, such as a guidance message, displayed guidance and direct receipt of a telephone message.

In certain embodiments, the system is useful for aiding those in transition from military service and exhibiting Post Traumatic Stress Disorder. Others aided by the system may have been incarcerated or have behavioral issues. The neural feedback that is detected in a setting of everyday life, i.e. in normal or daily routine settings is provided through the use of the mobile device. The system and other accompanying services may be provided by private companies, health care providers or networks to individuals in these types of transitions. In other embodiments, the system can be self-directed via pre-recorded instructions and used independently of a health care practitioner.

### Brief Description of the Embodiments

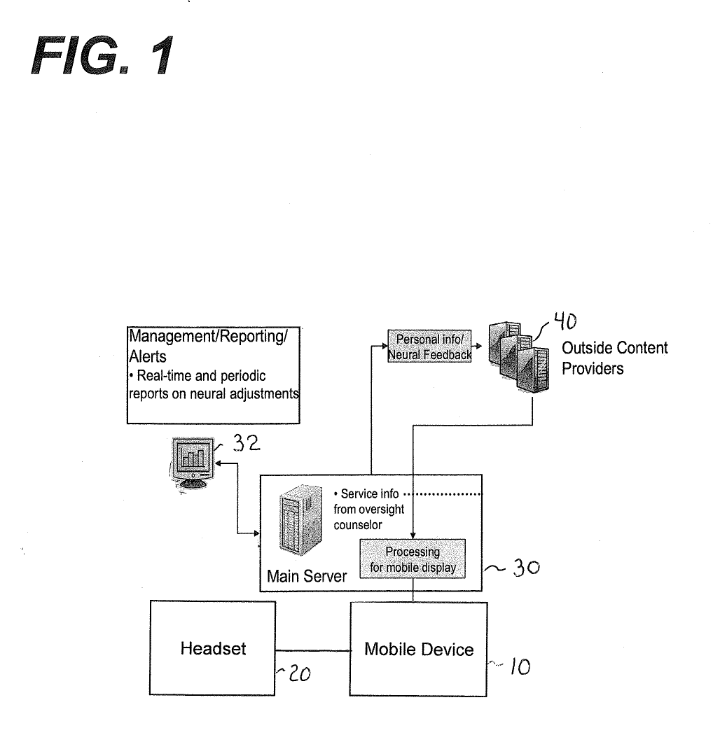

FIG. 1 is a diagram of the neural feedback system of the invention.

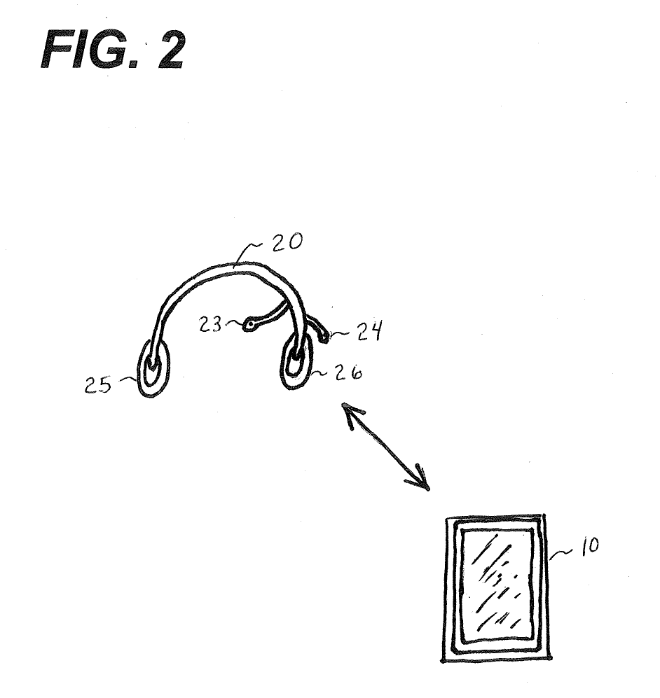

FIG. 2 is a diagram of a modified embodiment of the neural feedback system of the invention.

### DETAILED DESCRIPTION OF THE EMBODIMENTS

One embodiment of the invention is shown in the diagram of FIG. 1, which shows the neural feedback system of the invention.

A user, not shown, is provided with a portable electronic device or mobile device 10 that is, for example, a smart phone having a connection via cell phone service to other phones and to the Internet for data transmission. Although not shown, the mobile device has a microprocessor and memory that stores software for running an application on the phone. The portable electronic device may take various forms, such as, without limitation, a mobile phone, a GPS enabled mobile phone, a CDMA enabled mobile phone, a GPRS enabled mobile phone, a mobile phone with a camera, a mobile phone with browser capabilities, a GPS unit, a tracking unit, a portable electronic device built for this specific purpose, a laptop computer, a personal digital assistant, an MP3 player, a camera, a handheld device, a pager, a tablet device, and/or a portable gaming device. The portable electronic facility may also be wearable.

A user wears a headset 20 having electrodes 23 and 24, for example and optionally ear phones 25 and 26. The electrodes, which are typically positioned at the forehead of the user and behind or at the ear, detect brain waves. The detected brain waves are outputted to the mobile device through Bluetooth or a wired connection.

At the mobile device, the input data of the detected brainwaves are transmitted to a computer, server or storage device 30. The detected output of the brain waves may be processed at the computer and monitored by software running on the computer to make observations that are related to the mental state of the wearer of the headset, which can occur in real time.

Processing of the detected brain waves by the computer results in a determination being made as to whether or not the brain waves being detected indicate a situation in which the system should generate a response that is to be received by the user. For example, a given user's data can be stored in the computer and compared with real time data being inputted from the headsets and transmitted through the mobile device, for comparison purposes, to determine when a response should be generated. Also, the detected brain waves can be processed to exhibit a level of output that reflects the brain wave activity and, if the output level exceeds a preset or “normal” condition, a response can be generated by the system. The detected brain waves may also be monitored by a trained system operator at a terminal 32 connected to the computer, directly or remotely, e.g., through the internet.

When it is determined by the system software or by an operator who monitors the brain waves of the user through the terminal 32 that a response is to be generated, the response may take one or more forms. A response may simply provide an alert on the phone, which the user receives, and then takes appropriate action, such as selecting a mobile phone application and/or listening or reading for a display of the mobile device guidance messages.

The response may constitute a file or message personally tailored for the individual user in view of the user's known personal information and needs given the brain wave activity detected. Such personal information may be stored on the mobile phone 10, the computer 20 or may be obtained from an outside service provider 40. The intention is that the response is generated to suit the needs of the user of the system so that emotional or/and physical responses are elicited that favor the user's mental health and well being at the time the user is determined to be exhibiting brain wave activity, which is determined to require a response.

The mobile device may have specific user interface capability for the user, which does not require any information of a personal nature to be exchanged, so that the anonymity of the user is preserved even while the brain waves are being detected. In such a case, the brain waves can be monitored by the headset 20 and the detected brain waves transmitted to the computer 20 by without any identification information being determined by the computer.

Further, the user may have an application running on the mobile device as shown in FIG. 2 that receives the detected brain waves, processes the output of the headset and determines whether or not a response to the detected brain waves should be generated for the user, as feedback about the level of brain wave activity being detected. If a response is to be generated, then the same types of messages can be displayed, and the user may take appropriate action.

For example, the mobile device might display a version of the user interface based on the contents of a stored file, message or other contents. Also, the user can modify the mobile phone interface to provide suitable messages, guidance options for selection by the user. Any adaptable user interface may provide an appropriate response to the user such as an answer to a user's query.

These services can be brought into a health-related facility and/or delivered to the individual either through a local area network, through an internet delivery system, through cellular phone services, through any portable device or through software systems embedded in computer readable media. The menu or information regarding these neural feedback services can be made available in audio format and in several different languages, so that they are accessible to various subjects such as probationers, institutionalized individuals, and others who are illiterate and/or do not speak or read English.

The information can be provided to the user either electronically through a wireless internet or intranet connection to a the mobile device 10, or by transfer of electronic data to the mobile device 10 through a portable storage medium such as a portable disk, hard disk within a device, CD, DVD, memory stick or other memory device that is connected to the mobile device.

The computer 30 of the system may include one or more servers optionally including but not limited to an applications server that can be located within an institution, or facility or can be accessible by a terminal (32) in the institution or facility and by others within the correctional, justice, health, military or educational systems such as probation officers or case workers, for example, and is connected to one or more database servers containing the data to be provided to the subject.

The present disclosure is primarily directed to neural feedback through a mobile electronic device 10. The systems and methods should assist any individual in need of the device, including those in need of successful transition from a structured environment to a less structured living situation, such as society at large. It also includes the use of the device with populations to avoid institutionalization and it is not restricted to those who are in need of transition to the “outside”. In a preferred embodiment, the system is used by health care professionals, penal institutions, probation officers, inmates and parolees to reduce incarceration, health care facility admissions, recidivism, in which the systems and methods provide a comprehensive, individually tailored tool for providing support to avoid, or before and during the transition from living in an institution to living in free society. Besides prisons, correctional facilities and probationary periods, other examples of structured environments include mental and health institutions, military service or educational institutions. Potential subjects include prisoners, probationers, parolees, institutionalized individuals, troubled youth, mental health patients, immigrants, military personnel, students and others entering a new societal or alien environment or those who have not be in a structured environment yet and using it to increase their coping so as to avoid institutionalization. In preferred embodiments, the systems are directed to providing services to a user.

The central computer 30 of FIG. 1 can be connected to an intranet or internet network, including but not limited to the World Wide Web. The central computer can be located within an institution such as a prison, within a state or federally owned or controlled office or center, for example. The central computer includes a processor, software instructions, and includes or is connected to one or more memory storage devices and the ability to provide applications for neural feedback, and other technologically-based operant training techniques. The memory storage devices can include databases for storage of personal information about users and a particular user's release program such as information about goods and services available to and needed by a user during the transition period and targeted to a particular location.

Although FIG. 1 shows a mobile device 10 connectable to the central computer 30, either through a network such as an internet or intranet system, or directly through a disc drive, communication port or other connections known in the art, FIG. 2 shows an embodiment in which the mobile device 10 operates independently of the computer 30 at certain times, or all of the time, and therefore the combination of the headset 20 and the mobile device 10 can attain the objective of monitoring of the detected brain waves and the determination of when a response needs to be generated. In addition the appropriate response can be determined by the software application on the mobile phone 10 and the user can be prevented with the options of reponses available through the mobile device, such as the display of guidance or messages, the generation of sounds to be listened to through the headst or the prompting of receiving additional information from outside the mobile device, such as through the Internet.

The portable device 10 can include transmission and reception capability from satellites as well as through cellular services or wireless services. The device can also include a display device to display requested information to a user, and/or an audio speaker and audio drivers effective to provide audible information to a user. The devices also include software instructions for storing, accessing, and producing visual or audio communications, such as relaxing music and/or videos and other data and feedback needed for a user.

The computer 30 can or another outside server or computer can provide transportation information such as a map of the user's current location, social support, an alert service to alert others of a predetermined condition of the user as evidenced by the user's brain waves, and video displays displaying guidance information or calming information.

While preferred embodiments have been set forth with specific details, further embodiments, modifications and variations are contemplated according to the broader aspects of the present invention, all as determined by the spirit and scope of the following claims.

I claim:

1. A neural feedback system, comprising:
a headset having at least one electrode for detecting brain waves that is worn by a user;
a portable device having memory and a processor to which the headset is connected,
wherein the brain waves are monitored by the headset and processed by the processor running on the mobile device to output a measure of the brain waves that correlates with a user's level of brain activity

2. A neural feedback system according to claim 1, wherein the headset is wirelessly coupled to the mobile device.

3. A neural feedback system according to claim 1, wherein the headset includes earphones that provide predetermined messages, prompts or other media support via the mobile device in response to predetermined levels of brain wave activity.

4. A neural feedback system according to claim 1, wherein the mobile device is a smart phone that connects a remote party to the user in response to predetermined levels of brain wave activity.

5. A neural feedback system according to claim 1, wherein the mobile device displays messages to the user in response to predetermined levels of brain wave activity.

5. A neural feedback system according to claim 1, wherein the mobile device provides an alert to the user in response to predetermined levels of brain wave activity.

6. A neural feedback system according to claim 5, wherein the mobile device displays messages to the user and the user selects a response to the displayed messages in response to predetermined levels of brain wave activity.
